(19) United States
(12) Patent Application Publication
(10) Pub. No.: US 2026/0275306 A1
(43) Pub. Date: Sep. 17, 2026

(54) STRAND-DISPLACING DNA POLYMERASES

(71) Applicant: VARIGEN BIOSCIENCES CORPORATION, Middleton, WI (US)

(72) Inventors: Aaron Lomax, Madison, WI (US); Kurt Throckmorton, Middleton, WI (US); Yogesh Chander, Waunakee, WI (US); Alyssa Takara Bigelow Hassinger, Madison, WI (US); David Mead, Middleton, WI (US)

(21) Appl. No.: 19/566,437

(22) Filed: Mar. 13, 2026

Related U.S. Application Data
(60) Provisional application No. 63/771,767, filed on Mar. 14, 2025.

(51) Int. Cl. C12N 9/12; C12Q 1/6844

(52) U.S. Cl. CPC C12N 9/1252; C12N 9/1276; C07K 2319/21; C12Q 1/6844; C12Y 207/07007; C12Y 207/07049

(57) ABSTRACT

Provided herein are compositions and methods for use in biological assays. In particular, provided herein are DNA polymerases with enhanced strand-displacement activity for use in amplification assays such as, for example, loop-mediated isothermal amplification (LAMP) or recombinase polymerase amplification (RPA) assays.

### CROSS-REFERENCE TO RELATED APPLICATION

This application claims the benefit of U.S. Provisional Patent Application No. 63/771,767, filed Mar. 14, 2025, which is incorporated by reference herein in its entirety.

### FIELD

The text of the computer readable sequence listing filed herewith, titled “VARI-44785.202_SequenceListing.xml”, created Mar. 13, 2026, having a file size of 27,162 bytes, is hereby incorporated by reference in its entirety.

### FIELD

Provided herein are compositions and methods for use in a variety of assays including, but not limited to amplification assays. In particular, provided herein are modified DNA polymerases with enhanced strand-displacing activity at low-to-moderate temperatures that find use in isothermal amplification assays such as recombinase polymerase amplification (RPA) assays, rolling circle amplification (RCA), and loop-mediated isothermal amplification (LAMP) assays.

### BACKGROUND

Isothermal amplification methods have become an attractive alternative to polymerase chain reaction (PCR). Many isothermal amplification assays have sensitivity and specificity comparable to PCR, but do not require expensive thermocycler instrumentation. Accordingly, isothermal amplification methods find use in resource-limited laboratories. Common examples of isothermal amplification are RPA (recombinase polymerase amplification), RCA (rolling circle amplification), and LAMP (loop-mediated isothermal amplification). These assays use DNA polymerase enzymes having strand-displacing activity, which is an activity of the polymerase to displace downstream DNA during nucleic acid synthesis (see, e.g., U.S. Pat. No. 10,787,653 B2). Different types of isothermal amplification methods use different enzymes for amplifying nucleic acids. For example, RPA assays use three proteins (T4 UvsX (recombinase), T4 UvsY (recombinase loading factor), and GP32 (single-stranded binding protein)); a pair of primers; and a strand-displacing DNA polymerase from Bacillus subtilis (Bsu) or Staphylococcus aureus (Sau) to amplify nucleic acids at low temperatures (Li, et al., Review: a comprehensive summary of a decade development of the recombinase polymerase amplification. Analyst, 2019; 14:31-67). In RCA, phi29 DNA polymerase and random short oligos are used to amplify nucleic acids at low temperatures (Mohsen and Tool, The Discovery of Rolling Circle Amplification and Rolling Circle Transcription. Acc Chem Res. 2016; 49(11): 2540-2550). In LAMP, 4-6 DNA primers and a Bst (Bacillus sterothermophilus) DNA polymerase are employed to amplify target nucleic acid at high temperatures. Bst DNA polymerases (DNAPs) are commonly used for sequence amplification of DNA in a LAMP assay, but these polymerases amplify efficiently only at higher temperatures (60-72° C.) (Mori et al., Loop-mediated isothermal amplification (LAMP): a rapid, accurate, and cost-effective diagnostic method for infectious diseases. J. Infect. Chemother, 2009; 15: 62-69). phi29 DNA polymerase has not been used for sequence-specific DNA amplification because it produces off-target background noise. Bsu or Sau DNA polymerases have been used for RPA amplification at low temperatures (30-40° C.) but these enzymes have very weak strand displacement activity. Accordingly, a low temperature DNA polymerase with potent strand displacing activity is needed to improve assays like LAMP and RPA (Nam, et al., Low-Temperature Loop-Mediated Isothermal Amplification Operating at Physiological Temperature. Biosensesors, 2023; 13: 367-379).

### SUMMARY

Provided herein are compositions and methods for use in various assays including, but not limited to amplification assays. Provided herein are variant DNA polymerases with enhanced strand-displacement activity. Nucleotide and amino acid (polypeptide) sequences are provided for the variant DNA polymerases having enhanced strand-displacement activity described herein. The polymerases disclosed herein are useful, for example, for use in isothermal amplification of nucleic acids at low temperatures. In some embodiments, the variant DNA polymerases lack an exonuclease domain. That is, in some embodiments, a variant DNA polymerase is provided that comprises an amino acid sequence lacking an exonuclease domain (i.e., an “exonuclease-free” DNA polymerase). In some embodiments, a variant DNA polymerase disclosed herein comprises an amino acid sequence that is modified to include a poly-histidine tag (e.g., a tag comprising 5-15 (e.g., 5, 6, 7, 8, 9, 10, 11, 12, 13, 14, or 15) histidines) at the N-terminus of the variant DNA polymerase amino acid sequence.

In some embodiments, the DNA polymerase variant is a variant of the wild-type Sp-402 or Pb-439 DNA polymerases. In some embodiments, the Sp-402 or Pb-439 polymerase is modified to lack 5′-3′ exonuclease activity. In some embodiments, the Sp-402 or Pb-439 variant may lack 5′-3′ exonuclease activity and may further be modified to include a histidine tag at its N-terminus. According to specific, non-limiting embodiments, the polypeptide has an amino acid sequence selected from, for example, SEQ ID NOs: 6-7.

Further provided is a composition, reaction mixture, or kit comprising a variant polymerase or polypeptide as described herein.

Certain embodiments provide a method of amplifying a target nucleic acid, comprising a) contacting a sample with a polymerase, polypeptide, composition, or reaction mixture described herein; and b) performing an amplification assay on the sample.

Additional embodiments provide a method of detecting a target nucleic acid, comprising a) contacting a sample with a polymerase, polypeptide, composition, or reaction mixture described herein; and b) performing an amplification assay on the sample.

Further, provided herein is the use of a polymerase, polypeptide, composition, reaction mixture or kit as described herein to perform an amplification or other assay.

While much of the disclosure below is directed towards amplification assays, it will be understood that the present disclosure is not limited to a particular use or assay and that the polymerases, polypeptides, and nucleic acids herein may be useful in any number of biological or chemical reactions and that such reactions are contemplated by the present disclosure.

Additional embodiments are described herein.

### BRIEF DESCRIPTION OF THE DRAWINGS

FIG. 1: Phylogenetic relationship of the polymerase domains of various DNA Pol I enzymes including Sp-402 and Pb-439. Sequences were identified using BLAST and aligned, truncated to exclude the 5′-3′ exonuclease domain, and the tree constructed with Geobacillus stearothermophilus DNA Pol I (Bst1) as an outgroup in UGENE. The sequences were aligned with MAFFT and the tree constructed with FastTree using default parameters. See, e.g., Okonechnikov (2012) “Unipro UGENE: a unified bioinformatics toolkit” Bioinformatics. 28 (8): 1166-7; Katoh (2002) “MAFFT: a novel method for rapid multiple sequence alignment based on fast Fourier transform” Nucleic Acids Research 30 (14): 3059-66; and Price (2010) “FastTree 2—Approximately Maximum-Likelihood Trees for Large Alignments” PLoS ONE 5(3): e9490, each of which is incorporated herein by reference.

FIG. 2: Images of ethidium bromide-stained agarose gels of single-stranded DNA primer extension assays demonstrating robust strand-displacement activity of WT-Δexo-Sp-402 and WT-Δexo-Pb-439 compared to Sau DNA Polymerase I, large fragment (Intact Genomics, MO) and Bsu DNAP, large fragment (New England Biolabs, MA). The gel image indicates DNA-dependent DNA polymerization and strand-displacement of M13 phage DNA in single-stranded DNA primer-extension assays performed at various temperatures ranging between 30 to 60° C. The high molecular weight smearing and concentration of DNA in the gel well is indicative of strand-displacement activity of WT-Δexo-Sp-402 and WT-Δexo-Pb-439 relative to Sau DNAP, large fragment and Bsu DNAP, large fragment.

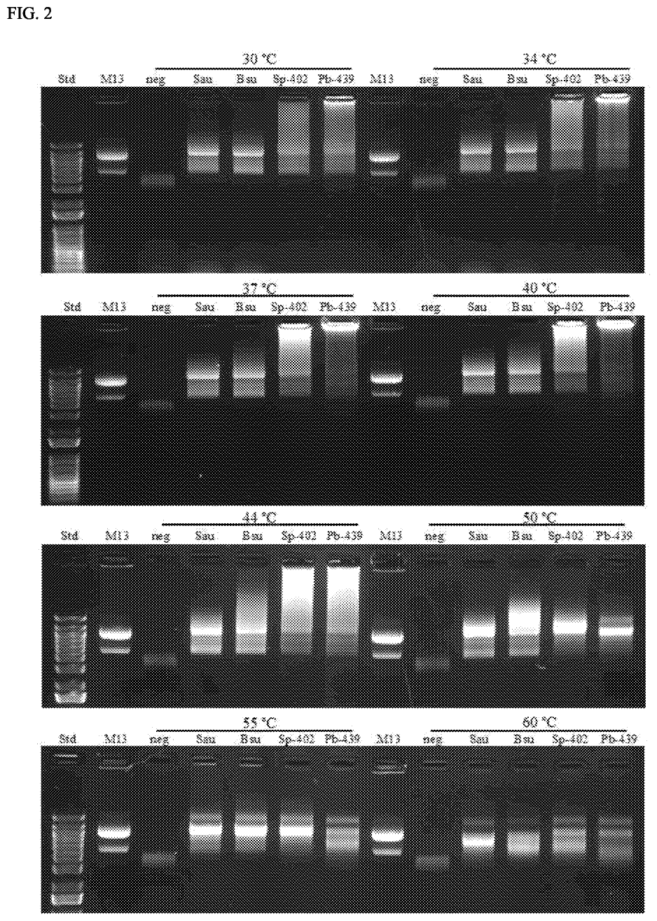

FIG. 3: Graphical depiction of DNA-dependent DNA polymerization activity of WT-Δexo-Sp-402 and WT-Δexo-Pb-439 compared to Bsu DNAP, large fragment (New England Biolabs, MA) in single-stranded M13 primer extension assays with Quant-IT PicoGreen (ThermoFisher). The graph shows the RFU formation rates of different dilutions of WT-Δexo-Sp-402 and WT-Δexo-Pb-439 relative to Sau DNAP, large fragment and Bsu DNAP, large fragment.

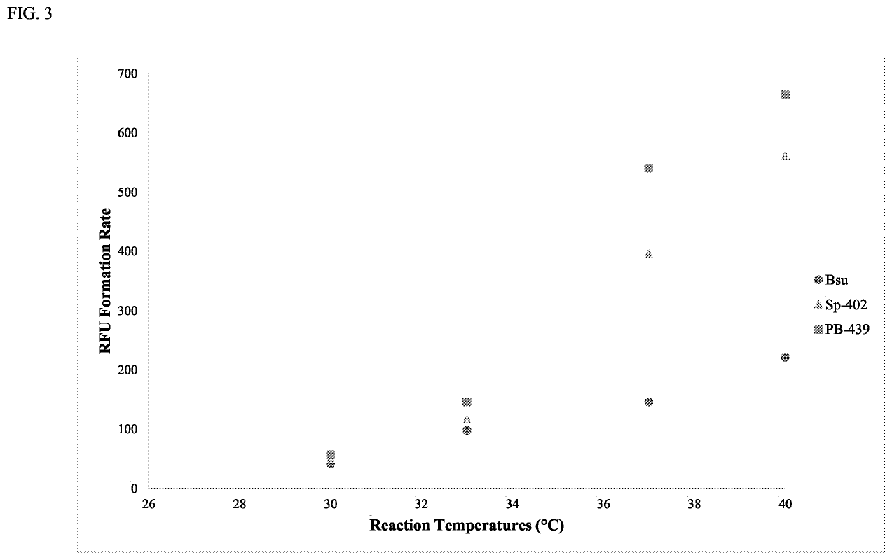

### DETAILED DESCRIPTION

Provided herein are compositions and methods for use in assays such as, but not limited to, nucleic acid amplification assays. In general, the present disclosure provides modified DNA polymerases (“DNAPs”) (and the associated polypeptide and nucleic acid sequences) with enhanced processivity and strand-displacement activity compared to the wild type versions of the DNA polymerases. According to some embodiments, the enhanced activity occurs during assays conducted at low-to-moderate temperatures (i.e. less than 60° C.). According to a specific embodiment, the DNA polymerases are modified from wild-type Sporosarcina psychrophila (Genbank Accession No. HJF33402.1) Sp-402 polymerase (“Sp-402”) and/or Psychrobacillus sp. (Genbank Accession No. WP_298471439.1) Pb-439 polymerase Pb-439 (“Pb-439”). As a specific example, the polymerases herein are useful in isothermal amplification assays such as RCA, RPA, and LAMP.

### Definitions

As used herein, the terms “about”, “approximately”, “substantially”, and “significantly” are understood by persons of ordinary skill in the art and will vary to some extent on the context in which they are used. If there are uses of these terms that are not clear to persons of ordinary skill in the art given the context in which they are used, “about” and “approximately” mean plus or minus less than or equal to 10% of the particular term and “substantially” and “significantly” mean plus or minus greater than 10% of the particular term.

As used herein, disclosure of ranges includes disclosure of all values and further divided ranges within the entire range, including endpoints and sub-ranges given for the ranges. As used herein, the disclosure of numeric ranges includes the endpoints and each intervening number therebetween with the same degree of precision. For example, for the range of 6-9, the numbers 7 and 8 are contemplated in addition to 6 and 9, and for the range 6.0-7.0, the numbers 6.0, 6.1, 6.2, 6.3, 6.4, 6.5, 6.6, 6.7, 6.8, 6.9, and 7.0 are explicitly contemplated.

As used herein, the suffix “-free” refers to an embodiment of the technology that omits the feature of the base root of the word to which “-free” is appended. That is, the term “X-free” as used herein means “without X”, where X is a feature of the technology omitted in the “X-free” technology. For example, a “calcium-free” composition does not comprise calcium, a “mixing-free” method does not comprise a mixing step, etc.

Although the terms “first”, “second”, “third”, etc. may be used herein to describe various steps, elements, compositions, components, regions, layers, and/or sections, these steps, elements, compositions, components, regions, layers, and/or sections should not be limited by these terms, unless otherwise indicated. These terms are used to distinguish one step, element, composition, component, region, layer, and/or section from another step, element, composition, component, region, layer, and/or section. Terms such as “first”, “second”, and other numerical terms when used herein do not imply a sequence or order unless clearly indicated by the context. Thus, a first step, element, composition, component, region, layer, or section discussed herein could be termed a second step, element, composition, component, region, layer, or section without departing from the disclosed teachings.

As used herein, the word “presence” or “absence” (or, alternatively, “present” or “absent”) is used in a relative sense to describe the amount or level of a particular entity (e.g., component, action, element). For example, when an entity is said to be “present”, it means the level or amount of this entity is above a pre-determined threshold; conversely, when an entity is said to be “absent”, it means the level or amount of this entity is below a pre-determined threshold. The pre-determined threshold may be the threshold for detectability associated with the particular test used to detect the entity or any other threshold. When an entity is “detected” it is “present”; when an entity is “not detected” it is “absent”.

As used herein, an “increase” or a “decrease” refers to a detectable (e.g., measured) positive or negative change, respectively, in the value of a variable relative to a previously measured value of the variable, relative to a pre-established value, and/or relative to a value of a standard control. An increase is a positive change preferably at least 10%, more preferably 50%, still more preferably 2-fold, even more preferably at least 5-fold, and most preferably at least 10-fold relative to the previously measured value of the variable, the pre-established value, and/or the value of a standard control. Similarly, a decrease is a negative change preferably at least 10%, more preferably 50%, still more preferably at least 80%, and most preferably at least 90% of the previously measured value of the variable, the pre-established value, and/or the value of a standard control. Other terms indicating quantitative changes or differences, such as “more” or “less,” are used herein in the same fashion as described above.

As used herein, “a” or “an” or “the” can mean one or more than one. For example, “a” cell can mean one cell or a plurality of cells.

The terms “protein” and “polypeptide” refer to compounds comprising amino acids joined via peptide bonds and are used interchangeably. A “protein” or “polypeptide” encoded by a gene is not limited to the amino acid sequence encoded by the gene, but includes post-translational modifications of the protein. Conventional one and three-letter amino acid codes are used herein as follows—Alanine: Ala, A; Arginine: Arg, R; Asparagine: Asn, N; Aspartate: Asp, D; Cysteine: Cys, C; Glutamate: Glu, E; Glutamine: Gln, Q; Glycine: Gly, G; Histidine: His, H; Isoleucine: Ile, I; Leucine: Leu, L; Lysine: Lys, K; Methionine: Met, M; Phenylalanine: Phe, F; Proline: Pro, P; Serine: Ser, S; Threonine: Thr, T; Tryptophan: Trp, W; Tyrosine: Tyr, Y; Valine: Val, V. As used herein, the codes Xaa and X refer to any amino acid.

It is well known that DNA (deoxyribonucleic acid) is a chain of nucleotides consisting of 4 types of nucleotides; A (adenine), T (thymine), C (cytosine), and G (guanine), and that RNA (ribonucleic acid) is comprised of 4 types of nucleotides; A, U (uracil), G, and C. It is also known that all of these 5 types of nucleotides specifically bind to one another in combinations called complementary base pairing. That is, adenine (A) pairs with thymine (T) (in the case of RNA, however, adenine (A) pairs with uracil (U)), and cytosine (C) pairs with guanine (G), so that each of these base pairs forms a double strand.

Where the term “amino acid sequence” is recited herein to refer to an amino acid sequence of a protein molecule, “amino acid sequence” and like terms such as “polypeptide” or “protein” are not meant to limit the amino acid sequence to the complete, native amino acid sequence associated with the recited protein molecule. Furthermore, an “amino acid sequence” can be deduced from the nucleic acid sequence encoding the protein.

The term “portion” when used in reference to a protein (as in “a portion of a given protein”) refers to fragments of that protein. The fragments may range in size from four amino acid residues to the entire amino sequence minus one amino acid (for example, the range in size includes 4, 5, 6, 7, 8, 9, 10, or more amino acids up to the entire amino acid sequence minus one amino acid).

The term “homolog” or “homologous” when used in reference to a polypeptide refers to a high degree of sequence identity between two polypeptides, or to a high degree of similarity between the three-dimensional structure, or to a high degree of similarity between the active site and the mechanism of action. In a preferred embodiment, a homolog has a greater than 60% sequence identity, and more preferably greater than 75% sequence identity, and still more preferably greater than 90% sequence identity, with a reference sequence.

As applied to polypeptides, the term “substantial identity” means that two peptide sequences, when optimally aligned, such as by the programs GAP or BESTFIT using default gap weights, share at least 80 percent sequence identity, preferably at least 90 percent sequence identity, more preferably at least 95 percent sequence identity or more (e.g., 99 percent sequence identity). Preferably, residue positions which are not identical differ by conservative amino acid substitutions.

The term “mutation” as used herein indicates any modification of a nucleic acid that results in an altered nucleic acid, e.g., that produces an amino acid “substitution” in a polypeptide (e.g., thus producing a “mutant” polypeptide or “mutant” nucleic acid). Mutations include, for example, point mutations, deletions, or insertions of single or multiple residues in a polynucleotide, which includes alterations arising within a protein-encoding region of a gene as well as alterations in regions outside of a protein-encoding sequence, such as, but not limited to, regulatory or promoter sequences. A genetic alteration may be a mutation of any type. For instance, the mutation may constitute a point mutation, a frame-shift mutation, an insertion, or a deletion of part or all of a gene. In addition, in some embodiments of the modified microorganism, a portion of the microorganism genome has been replaced with a heterologous polynucleotide. In some embodiments, the mutations are naturally-occurring. In other embodiments, the mutations are the results of artificial mutation pressure. In still other embodiments, the mutations in the microorganism genome are the result of genetic engineering.

The terms “variant” and “mutant” when used in reference to a polypeptide refer to an amino acid sequence that differs by one or more amino acids from another, usually related polypeptide. The variant may have “conservative” changes, wherein a substituted amino acid has similar structural or chemical properties. One type of conservative amino acid substitutions refers to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, and asparagine-glutamine. More rarely, a variant may have “non-conservative” changes (e.g., replacement of a glycine with a tryptophan). Similar minor variations may also include amino acid deletions or insertions (i.e., additions), or both. Guidance in determining which and how many amino acid residues may be substituted, inserted or deleted without abolishing biological activity may be found using computer programs well known in the art, for example, DNAStar software. Variants can be tested in functional assays. Preferred variants have less than 10%, and preferably less than 5%, and still more preferably less than 2% changes (e.g., substitutions, deletions, insertions, etc.) relative to another reference sequence. In some embodiments, the reference sequence is a wild-type sequence.

The term “domain” when used in reference to a polypeptide refers to a subsection of the polypeptide which possesses a unique structural and/or functional characteristic; typically, this characteristic is similar across diverse polypeptides. The subsection typically comprises contiguous amino acids, although it may also comprise amino acids which act in concert or which are in close proximity due to folding or other configurations. Examples of a protein domain include the transmembrane domains, and the glycosylation sites.

The term “gene” refers to a nucleic acid (e.g., DNA or RNA) sequence that comprises coding sequences necessary for the production of an RNA, or a polypeptide or its precursor. A functional polypeptide can be encoded by a full length coding sequence or by any portion of the coding sequence as long as the desired activity or functional properties (e.g., enzymatic activity, ligand binding, signal transduction, etc.) of the polypeptide are retained. The term “portion” when used in reference to a gene refers to fragments of that gene. The fragments may range in size from a few nucleotides to the entire gene sequence minus one nucleotide. Thus, “a nucleotide comprising at least a portion of a gene” may comprise fragments of the gene or the entire gene.

The term “gene” also encompasses the coding regions of a structural gene and includes sequences located adjacent to the coding region on both the 5′ and 3′ ends for a distance of about 1 kb on either end such that the gene corresponds to the length of the full-length mRNA. The sequences which are located 5′ of the coding region and which are present on the mRNA are referred to as 5′ non-translated sequences. The sequences which are located 3′ or downstream of the coding region and which are present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene which are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

In addition to containing introns, genomic forms of a gene may also include sequences located on both the 5′ and 3′ end of the sequences which are present on the RNA transcript. These sequences are referred to as “flanking” sequences or regions (these flanking sequences are located 5′ or 3′ to the non-translated sequences present on the mRNA transcript). The 5′ flanking region may contain regulatory sequences such as promoters and enhancers which control or influence the transcription of the gene. The 3′ flanking region may contain sequences which direct the termination of transcription, posttranscriptional cleavage and polyadenylation.

The terms “oligonucleotide” or “polynucleotide” or “nucleotide” or “nucleic acid” refer to a molecule comprised of two or more deoxyribonucleotides or ribonucleotides, preferably more than three, and usually more than ten. The exact size will depend on many factors, which in turn depends on the ultimate function or use of the oligonucleotide. The oligonucleotide may be generated in any manner, including chemical synthesis, DNA replication, reverse transcription, or a combination thereof. The terms encompass sequences that include any of the known base analogs of DNA and RNA including, but not limited to, 4 acetylcytosine, 8-hydroxy-N6-methyladenosine, aziridinylcytosine, pseudoisocytosine, 5 (carboxyhydroxylmethyl) uracil, 5-fluorouracil, 5 bromouracil, 5-carboxymethylaminomethyl 2 thiouracil, 5 carboxymethyl-aminomethyluracil, dihydrouracil, inosine, N6 isopentenyladenine, 1 methyladenine, 1-methylpseudo¬uracil, 1 methylguanine, 1 methylinosine, 2,2-dimethyl¬guanine, 2 methyladenine, 2 methylguanine, 3-methyl¬cytosine, 5 methylcytosine, N6 methyladenine, 7 methylguanine, 5 methylaminomethyluracil, 5-methoxy-amino-methyl 2 thiouracil, beta D mannosylqueosine, 5′-methoxycarbonylmethyluracil, 5 methoxyuracil, 2 methylthio N6 isopentenyladenine, uracil 5 oxyacetic acid methylester, uracil 5 oxyacetic acid, oxybutoxosine, pseudouracil, queosine, 2 thiocytosine, 5-methyl-2 thiouracil, 2-thiouracil, 4 thiouracil, 5-methyluracil, N-uracil 5 oxyacetic acid methylester, uracil 5 oxyacetic acid, pseudouracil, queosine, 2-thiocytosine, and 2,6 diaminopurine.

As used here, the term “polymerase” or “DNA polymerase” refers to an enzyme that synthesizes a new copy of a nucleic acid strand using a parent strand as a template. In general, DNA polymerase transfers the phosphoryl group to the 3′OH-end of a growing DNA strand, one nucleotide at a time. The 3′—OH group acts as a nucleophile and attacks the incoming deoxyribonucleoside triphosphate at the α-phosphorus, forming a phosphodiester bond and releasing inorganic phosphate. DNA polymerase needs a template strand and a short RNA oligonucleotide primer to initiate replication by adding nucleotides only at the 3′ end of the growing chain. Since DNA polymerase moves in the 5′-3′ direction, replication always occurs in the same direction. The 3′-5′ exonuclease function of the polymerase removes incorrectly added nucleotides during replication, preventing DNA mutation. During proofreading, DNA polymerase checks each added nucleotide after incorporating it and removes it if there is a mismatch through its 3′-5′ exonuclease function.

As used herein, the terms “strand displacement” or “strand displacement activity” is a process in which a DNA polymerase displaces the newly produced DNA strand and continues its strand elongation, generating a newly synthesized single-stranded DNA template.

As used herein, the term “amplification” refers to a process by which multiple copies of a nucleic acid template are made via interaction with a DNA polymerase. Multiple rounds of amplification may result in the production of a high number of copies of the original template. For example, the amount of the template in the original sample may be so small as to be undetectable via a specific detection method, but after amplification, the amount of the template may be at detectable levels for the specific detection method.

For the purposes of the present disclosure, the term “low temperature” in relation to amplification refers to temperatures below 40° C., The term “moderate temperature” in relation to amplification refers to temperatures between 4° and 60° C. The term “high temperature” in relation to amplification refers to temperatures above 60° C.

As used herein, the term “primer” refers to an oligonucleotide, whether occurring naturally as in a purified restriction digest or produced synthetically, that is capable of acting as a point of initiation of synthesis when placed under conditions in which synthesis of a primer extension product that is complementary to a nucleic acid strand is induced, (i.e., in the presence of nucleotides and an inducing agent such as DNA polymerase and at a suitable temperature and pH). The primer is preferably single stranded for maximum efficiency in amplification but may alternatively be double stranded. If double stranded, the primer is first treated to separate its strands before being used to prepare extension products. Preferably, the primer is an oligodeoxyribonucleotide. The primer must be sufficiently long to prime the synthesis of extension products in the presence of the inducing agent. The exact lengths of the primers will depend on many factors, including temperature, source of primer and the use of the method.

The following terms are used to describe the sequence relationships between two or more polynucleotides: “reference sequence”, “sequence identity”, “percentage of sequence identity”, and “substantial identity”. A “reference sequence” is a defined sequence used as a basis for a sequence comparison; a reference sequence may be a subset of a larger sequence, for example, as a segment of a full-length cDNA sequence given in a sequence listing or may comprise a complete gene sequence. Generally, a reference sequence is at least 20 nucleotides in length, frequently at least 25 nucleotides in length, and often at least 50 nucleotides in length. Since two polynucleotides may each (1) comprise a sequence (i.e., a portion of the complete polynucleotide sequence) that is similar between the two polynucleotides, and (2) may further comprise a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window”, as used herein, refers to a conceptual segment of at least 20 contiguous nucleotide positions wherein a polynucleotide sequence may be compared to a reference sequence of at least 20 contiguous nucleotides and wherein the portion of the polynucleotide sequence in the comparison window may comprise additions or deletions (i.e., gaps) of 20 percent or less as compared to the reference sequence (which does not comprise additions or deletions) for optimal alignment of the two sequences. Optimal alignment of sequences for aligning a comparison window may be conducted by the local homology algorithm of Smith and Waterman [Smith and Waterman, Adv. Appl. Math. 2: 482 (1981)] by the homology alignment algorithm of Needleman and Wunsch [Needleman and Wunsch, J. Mol. Biol. 48:443 (1970)], by the search for similarity method of Pearson and Lipman [Pearson and Lipman, Proc. Natl. Acad. Sci. (U.S.A.) 85:2444 (1988)], by computerized implementations of these algorithms (GAP, BESTFIT, FASTA, and TFASTA in the Wisconsin Genetics Software Package Release 7.0, Genetics Computer Group, 575 Science Dr., Madison, Wis.), or by inspection, and the best alignment (i.e., resulting in the highest percentage of homology over the comparison window) generated by the various methods is selected. The term “sequence identity” means that two polynucleotide sequences are identical (i.e., on a nucleotide-by-nucleotide basis) over the window of comparison. The term “percentage of sequence identity” is calculated by comparing two optimally aligned sequences over the window of comparison, determining the number of positions at which the identical nucleic acid base (e.g., A, T, C, G, U, or I) occurs in both sequences to yield the number of matched positions, dividing the number of matched positions by the total number of positions in the window of comparison (i.e., the window size), and multiplying the result by 100 to yield the percentage of sequence identity. The terms “substantial identity” as used herein denotes a characteristic of a polynucleotide sequence, wherein the polynucleotide comprises a sequence that has at least 85 percent sequence identity, preferably at least 90 to 95 percent sequence identity, more usually at least 99 percent sequence identity as compared to a reference sequence over a comparison window of at least 20 nucleotide positions, frequently over a window of at least 25-50 nucleotides, wherein the percentage of sequence identity is calculated by comparing the reference sequence to the polynucleotide sequence which may include deletions or additions which total 20 percent or less of the reference sequence over the window of comparison. The reference sequence may be a subset of a larger sequence, for example, as a segment of the full-length sequences of the compositions claimed in the present invention.

The term “substantially homologous” when used in reference to a double-stranded nucleic acid sequence such as a cDNA or genomic clone refers to any probe that can hybridize to either or both strands of the double-stranded nucleic acid sequence under conditions of low to high stringency as described above.

The term “substantially homologous” when used in reference to a single-stranded nucleic acid sequence refers to any probe that can hybridize (i.e., it is the complement of) the single-stranded nucleic acid sequence under conditions of low to high stringency as described above.

The term “wild-type” when made in reference to a nucleotide sequence, amino acid sequence, protein, enzyme, gene or object or a product therefrom, refers to a nucleotide sequence, amino acid sequence, protein, enzyme, gene or object or a product therefrom that has the characteristics of the nucleotide sequence, amino acid sequence, protein, enzyme, gene or object or a product therefrom isolated from a naturally occurring source. The term “naturally-occurring” as applied to an object refers to the fact that an object can be found in nature. For example, a polypeptide or polynucleotide sequence that is present in an organism (including viruses) that can be isolated from a source in nature and which has not been intentionally modified by man in the laboratory is naturally-occurring. A wild-type gene is frequently that gene which is most frequently observed in a population and is thus arbitrarily designated the “normal” or “wild-type” form of the gene. In contrast, the term “mutant” when made in reference to a gene or to a gene product refers, respectively, to a gene or to a gene product which displays modifications in sequence and/or functional properties (i.e., altered characteristics) when compared to the wild-type gene or gene product. It is noted that naturally-occurring mutants can be isolated; these are identified by the fact that they have altered characteristics when compared to the wild-type gene or gene product. For the purposes of the present disclosure, the term “modified” refers to a nucleotide sequence, amino acid sequence, protein, enzyme, gene or object or a product therefrom which has been intentionally modified in a laboratory or other environment and is thus, non-naturally-occurring.

The terms “variant” and “mutant” when used in reference to a nucleotide (or amino acid) sequence refer to a nucleic acid (or amino acid) sequence that differs by one or more nucleotides (or amino acids) from another, usually related, nucleotide (or amino acid) sequence. A “variation” is a difference between two different sequences; typically, one sequence is a reference sequence.

The term “detection assay” refers to an assay for detecting the presence or absence of a wild-type or variant nucleic acid sequence (e.g., mutation or polymorphism) in a given allele of a particular gene, or for detecting the presence or absence of a particular protein or the activity or effect of a particular protein or for detecting the presence or absence of a variant of a particular protein.

The term “sample” is used in its broadest sense. In one sense it can refer to an animal cell or tissue. In another sense, it is meant to include a specimen or culture obtained from any source, as well as biological and environmental samples. Biological samples may be obtained from plants or animals (including humans) and encompass fluids, solids, tissues, and gases. Environmental samples include environmental material such as surface matter, soil, water, and industrial samples. These examples are not to be construed as limiting the sample types applicable to the present invention.

As used herein, the term “histidine tag” refers to an amino acid sequence of multiple histidine amino acids that is appended to another polypeptide. A histidine tag may also be called a “polyhistidine tag”. In some embodiments, a histidine tag comprises 2 to 15 histidines (e.g., 2, 3, 4, 5, 6, 7, 8, 9, 10, 11, 12, 13, 14, or 15 histidines). A histidine tag may be appended to the C-terminus or to the N-terminus of another polypeptide. A histidine tag finds use in affinity purification of a protein comprising the histidine tag using a nickel affinity column. See, e.g., Hochuli (1988) “Genetic Approach to Facilitate Purification of Recombinant Proteins with a Novel Metal Chelate Adsorbent” Nat Biotechnol 6: 1321-25.

The present disclosure is not limited to the specific DNA polymerase variants presented here. Any number of suitable variants may be utilized. While certain aspects of the disclosure are illustrated with variants of wild-type Sp-402 or Pb-439 DNAPs, additional DNAPs are specifically contemplated herein and thus the disclosure herein should be considered to be exemplary of the types of modifications that can be made to known DNAPs with the herein described modifications to Sp-402 and Pb-439 being considered exemplary, non-limiting examples.

In some embodiments, the DNA polymerase variants of the present disclosure comprise one or more mutations relative to a full-length, wild-type Sporosarcina psychrophila (Genbank Accession No. HJF33402.1) Sp-402 polymerase (SEQ ID NO: 1), alternatively referred to as “Sp-402 DNAP,” or “Sp-402” or wild-type Psychrobacillus sp. (Genbank Accession No. WP_298471439.1) Pb-439 polymerase (SEQ ID NO: 2), alternatively referred to as “Pb-439 DNAP” or “Pb-439.”

In some embodiments, a DNA polymerase variant comprises one or more modifications to the full-length, wild-type nucleic acid polymerase. In some embodiments, such modifications optimize the full-length, wild-type polymerase for certain amplification (e.g., LAMP, RT-LAMP) and/or purification (e.g., Ni-affinity column protein purification) methods or conditions. In some embodiments, the 5′ to 3′ exonuclease domain of the full-length, wild-type, polymerase located at the N-terminus of the wild-type proteins is deleted. In some embodiments, a histidine tag is added to the N-terminus of the 5′ to 3′ exonuclease domain-deficient polymerase. In some embodiments, the histidine tag is a ten-histadine tag. In some embodiments, the ten-histidine tag comprises an amino acid sequence as shown in SEQ ID NO: 5.

According to specific exemplary embodiments, the DNA polymerase is a variant of the wild-type Sp-402 or Pb-439 polymerase. The full length wt Sp-402 polymerase amino acid sequence is shown in Table 1 as SEQ ID NO: 1. The full length wt PB-439 polymerase is shown in Table 1 as SEQ ID NO: 2. The 5′ to 3′ exonuclease domains of the wild-type Sp-402 and Pb-439 polymerases are shown in Table 1 as SEQ. ID NOs. 3 and 4.

According to various embodiments, the variant Sp-402 or Pb-439 polymerase lacks the 5′ to 3′ exonuclease domain located at the N-terminus of the wild-type version. In some embodiments, histidine tag is added to the N-terminus of the 5′ to 3′ exonuclease domain-deficient Sp-402 or Pb-439, polymerase.

Accordingly, in some embodiments the Sp-402 or Pb-439 polymerase variant is or comprises an amino acid sequence as shown in Table 1, SEQ ID NO: 6 (referred to herein as “WT-Δexo-Sp-402”) or SEQ ID NO: 7 (referred to herein as “WT-Δexo-Pb-439” polymerase), respectively.

TABLE 1
Amino acid sequences of FL-WT-Bst496 DNA polymerase (DNAP) and its
variants

| SEQ | | |
|---|---|---|
| ID NO | Description | Sequence |
| 1 | FL-WT-Sp- | MTTKKIVLLDGNSLAYRAFFALPLLTNDNGIHTNAVYGFTMML |
| | 402 DNAP | QNILEEEKPTHILVAWDAGKTTFRHKTYGEYKGGRQKTPPELSE |
| | | QFPYLRKLLDAYNIPQYELDQYEADDIIGTLSKTGDADGSEIVVIS |
| | | GDKDLTQLASSNTTVFITRKGMTDIEKYTPEHVMEKYGIEPRQII |
| | | DMKGLMGDASDNIPGVPGVGEKTALKLLVEYGSIENVYKSLDKI |
| | | TAKKLNQNLTENQEQAFMSKDLATIEVAAPITVSLNDLAYEGPD |
| | | MEEVTSIYRELKFKTLLDKIAPQAAEEPKEAIDVTIVSELMDADL |
| | | SNEMAIHVEMMDENYLTAEILGISLADEKNTLFVPLEVAKECVA |
| | | FKNWLQNTDKLKYTSDSKAAFASLARQGMEAEGFQFDLLLATY |
| | | IVNPSTTYTDVASVAGEFGYTEVEHDEVIYGKGAKKAAPAQEMI |
| | | AEHAGRKAQAIWKLRPDIEQKLKENDQYDLYHELELPLAAILGK |
| | | METTGVMTDRETLLTIGSELTAKLATLETTIYEIAGEKFNINSPKQ |
| | | LGVILFEKIGLTPIKKTKTGYSTAADVLEKLESEHEIISHILNYRQL |
| | | GKLNSTYIEGLLKEIFEDGKIHTRFQQALTTTGRLSSINPNLQNIPV |
| | | RLEEGRKIRAAFVPSEPGWYLFAADYSQIELRVLAHISQDEKLIE |
| | | AFREGADIHTKTAMDVFGVDNDSVTSDMRRAAKAVNFGIVYGI |
| | | SDYGLSQSLGITRKDAAKFIDTYLSSFPGVKQYMSDSVAEAKIKG |
| | | YVTTLMNRRRYLPEITSSNFNLRSFAERTAMNTPIQGSAADIIKK |
| | | AMIEMAKRLEADGMQTRMLLQVHDELIFEAPEDEIEKLKVIVPE |
| | | VMESALALDVPLKVDWAFGLSWYDTK |
| 2 | FL-WT-Pb- | MSSEKILLLDGNSLAYRAFFALPLLTNEHGIHTNAVYGFTMMLQ |
| | 439 DNAP | KIMDEENPTHMLVAFDAGKTTFRHSTFGEYKGGRQKTPPELSEQ |
| | | FPYLRKLIDAYGIKRYELEMYEADDIIGTLSRQAEKKGQQVVIVS |
| | | GDKDLTQLATDSTTVYITRKGITDIEKYTPEHVQEKYGLTPLQIID |
| | | MKGLMGDSSDNIPGVPGVGEKTAIKLLKEHGSVEQLYQALDSVS |
| | | GAKLKEKLIANEEQAKMSKELATIEINAPIDVSLEDITYSGPNMD |
| | | DVISIWKELSFKTLLEKTEYVAEEHSTTEMTFEIVENVNSSFLVDE |
| | | MSLHLELYDEQYHRADLLGIALSDGEKSYFIPGETAFQSKEFCA |
| | | WLEDESKIKYVVDSKATQAVSRKHGVSIAGVEFDLTLAAYIDNA |
| | | SLTADDVATIGKEYGYYNVLSNEQIYGKGAKKSVPEQEKLAEH |
| | | ATRKALALWHLKPILEKKLKENEQFKLYKELELPLASILGTMES |
| | | DGVKVDKQVLVEMGHDLEIKLKQIEGDIHALAGEAFNINSPKQL |
| | | GVILFEKLGLPPIKKTKTGYSTAADVLEKLASEHEIIEQILMYRQL |
| | | GKLNSTYIEGLLKEIHESDGKIHTRYQQALTTTGRLSSINPNLQNI |
| | | PIRLEEGRKIRKAFVPSNPEWVMFAADYSQIELRVLAHMAEDAN |
| | | LVEAFKNNMDIHTKTAMDVFHVEADAVDSNMRRAAKAVNFGI |
| | | VYGISDYGLSQSLDITRKEAAIFIEKYLKSFPGVKEYMDDSIQTAK |
| | | QKGFVTTILNRRRYLPEITSSNFNLRSFAERTAMNTPIQGSAADII |
| | | KKAMIDMAARLKKENMQTKMLLQVHDELIFEAPPEEIALLEKIV |
| | | PEVMESAIELNVPLKVEYAFGSSWYDTK |
| 3 | Exo, | MTTKKIVLLDGNSLAYRAFFALPLLTNDNGIHTNAVYGFTMML |
| | Exonuclease | QNILEEEKPTHILVAWDAGKTTFRHKTYGEYKGGRQKTPPELSE |
| | domain 402 | QFPYLRKLLDAYNIPQYELDQYEADDIIGTLSKTGDADGSEIVVIS |
| | | GDKDLTQLASSNTTVFITRKGMTDIEKYTPEHVMEKYGIEPRQII |
| | | DMKGLMGDASDNIPGVPGVGEKTALKLLVEYGSIENVYKSLDKI |
| | | TAKKLNQNLTENQEQAFMSKDLATIEVAAPITVSLNDLAYEGPD |
| | | MEEVTSIYRELKFKTLLDKIAPQAAEE |
| 4 | Exo, | MSSEKILLLDGNSLAYRAFFALPLLTNEHGIHTNAVYGFTMMLQ |
| | Exonuclease | KIMDEENPTHMLVAFDAGKTTFRHSTFGEYKGGRQKTPPELSEQ |
| | doman 439 | FPYLRKLIDAYGIKRYELEMYEADDIIGTLSRQAEKKGQQVVIVS |
| | | GDKDLTQLATDSTTVYITRKGITDIEKYTPEHVQEKYGLTPLQIID |
| | | MKGLMGDSSDNIPGVPGVGEKTAIKLLKEHGSVEQLYQALDSVS |
| | | GAKLKEKLIANEEQAKMSKELATIEINAPIDVSLEDITYSGPNMD |
| | | DVISIWKELSFKTLLEKTEYVAEEH |
| 5 | 10XHis, tag | MHHHHHHHHHH |
| | for nickel | |
| | affinity | |
| | purification | |
| 6 | WT-Aexo- | MPKEAIDVTIVSELMDADLSNEMAIHVEMMDENYLTAEILGISL |
| | Sp-402 | ADEKNTLFVPLEVAKECVAFKNWLQNTDKLKYTSDSKAAFASL |
| | DNAP | ARQGMEAEGFQFDLLLATYIVNPSTTYTDVASVAGEFGYTEVEH |
| | | DEVIYGKGAKKAAPAQEMIAEHAGRKAQAIWKLRPDIEQKLKE |
| | | NDQYDLYHELELPLAAILGKMETTGVMTDRETLLTIGSELTAKL |
| | | ATLETTIYEIAGEKFNINSPKQLGVILFEKIGLTPIKKTKTGYSTAA |
| | | DVLEKLESEHEIISHILNYRQLGKLNSTYIEGLLKEIFEDGKIHTRF |
| | | QQALTTTGRLSSINPNLQNIPVRLEEGRKIRAAFVPSEPGWYLFA |
| | | ADYSQIELRVLAHISQDEKLIEAFREGADIHTKTAMDVFGVDNDS |
| | | VTSDMRRAAKAVNFGIVYGISDYGLSQSLGITRKDAAKFIDTYLS |
| | | SFPGVKQYMSDSVAEAKIKGYVTTLMNRRRYLPEITSSNFNLRSF |
| | | AERTAMNTPIQGSAADIIKKAMIEMAKRLEADGMQTRMLLQVH |
| | | DELIFEAPEDEIEKLKVIVPEVMESALALDVPLKVDWAFGLSWY |
| | | DTK |
| 7 | WT-Aexo- | MSTTEMTFEIVENVNSSFLVDEMSLHLELYDEQYHRADLLGIAL |
| | Pb-439 | SDGEKSYFIPGETAFQSKEFCAWLEDESKIKYVVDSKATQAVSR |
| | DNAP | KHGVSIAGVEFDLTLAAYIDNASLTADDVATIGKEYGYYNVLSN |
| | | EQIYGKGAKKSVPEQEKLAEHATRKALALWHLKPILEKKLKENE |
| | | QFKLYKELELPLASILGTMESDGVKVDKQVLVEMGHDLEIKLKQ |
| | | IEGDIHALAGEAFNINSPKQLGVILFEKLGLPPIKKTKTGYSTAAD |
| | | VLEKLASEHEIIEQILMYRQLGKLNSTYIEGLLKEIHESDGKIHTR |
| | | YQQALTTTGRLSSINPNLQNIPIRLEEGRKIRKAFVPSNPEWVMF |
| | | AADYSQIELRVLAHMAEDANLVEAFKNNMDIHTKTAMDVFHV |
| | | EADAVDSNMRRAAKAVNFGIVYGISDYGLSQSLDITRKEAAIFIE |
| | | KYLKSFPGVKEYMDDSIQTAKQKGFVTTILNRRRYLPEITSSNFN |
| | | LRSFAERTAMNTPIQGSAADIIKKAMIDMAARLKKENMQTKML |
| | | LQVHDELIFEAPPEEIALLEKIVPEVMESAIELNVPLKVEYAFGSS |
| | | WYDTK |

In some embodiments, a Sp-402 and Pb-439 polymerase variant of the disclosure comprises a polypeptide having at least 90% (e.g., at least 90%, at least 91%, at least 92%, at least 93%, at least 94%, at least 95%, at least 96%, at least 97%, at least 98%, at least 99%, or about 100%) sequence identity to an amino acid sequence as shown in any of SEQ ID NOs: 6-7.

As stated above, the present disclosure further provides nucleic acid sequences encoding the variants of Sp-402 and Pb-439 as described herein. In some embodiments, the nucleic acid sequences are selected from SEQ ID NOs: 9-10.

Aspects of the disclosure relate to modified DNA polymerases suitable for use in an amplification method, for example, an isothermal amplification method, which amplifies nucleic acids of interest by incubating a reaction mix at a constant temperature for a certain amount of time. Examples of isothermal amplification methods include single-stranded (ss) M13 primer extension assays and Loop-mediated isothermal amplification. In some embodiments, the amplification method is just one step in another assay, for example, a nucleotide sequencing method.

In some embodiments, the modified DNA polymerases and their variants disclosed herein find use in single-stranded (ss) M13 primer extension assays. ssM13 primer extension assays use a primer that binds ssM13 which allows for the amplification of a complementary DNA strand. In some embodiments, primer extension assays involve running a reaction at a constant temperature ranging from 30 to 50° C. In some embodiments, the reaction may be run for a duration of between 15 minutes and 2 hours. In some embodiments the reaction is run for 1 hour. Commercially available DNAPs suitable for use in ssM13 primer extension assays include Bsu DNAP, Large Fragment (New England Biolabs, Cat. No. M0330 7), or Sau DNA Polymerase I, Large Fragment (Intact Genomics, Cat. No. 3595).

In some embodiments, the ssM13 primer extension assay involves the use of a DNAP having strand-displacement activity but lacking 5′-3′ exonuclease activity. In some embodiments, the modified DNAPs are selected from, for example, the wild-type Sp-402 and Pb-439 DNAP modified to include a histidine tag at its N-terminus but lacking 5′-3′ exonuclease activity such as, though not limited to, (WT-Δexo-Sp-402 DNAP; WT-Δexo-Pb-439 DNAP; SEQ ID NO: 6 and 7),

As stated above, the modified DNAPs of the present disclosure are useful in RT-LAMP for amplification of RNA. As described by Notomi et al., (2000), LAMP employs a set of primers (F3 primer, B3 primer, FIP, and BIP), that are specific for amplification of target nucleic acid (e.g., DNA or RNA). In addition, two more primers, Forward loop (FL) and Backword loop (BL), can be included in the reaction mix to speed up the time to results (in minutes) and improve LAMP assay performance.

LAMP primers specific for a given target nucleic acid (DNA/RNA) can be designed using software e.g., [PrimerExplorer (primerexplorer.jp/e); NEB LAMP primer design tool (lamp.neb.com/#!/); LAVA (Torres et al, 2011); LAMP Designer (www.premierbiosoft.com/isothermal/lamp.html)].

LAMP is an isothermal method of amplification that involves running a reaction at a constant temperature typically ranging from 60° C.-72° C. for a duration of time, often ranging 85 to 100 minutes (Notomi et al., 2000). The new DNA polymerases of the present disclosure enable LAMP reactions at much lower temperatures including, for example, equal to or less than 44° C., equal to or less than 37° C., or equal to or less than 30° C.

According to an embodiment, RT-LAMP involves use of a modified DNAP having strand displacement activity but lacking 5′-3′ exonuclease activity such as, but not limited to, the modified Sp-402 and Pb-439 DNA polymerases disclosed herein including those shown in SEQ ID NOs: 6 and 7, and a secondary enzyme, RT polymerase (also known as, Reverse Transcriptase (RT)). Enzymes having reverse transcriptase activity are described in, for example, Kati et al., (1992); J. Biol. Chem., 267(36): 25988-97; Kotewicz et al., (1985); and Gene, 35(3): 249-58); and include, for example, WARMSTART RTx (New England Biolabs, Cat. No. M0380); SuperScript IV (ThermoFisher Scientific, Cat. No. 18090010); and M-MLV (ThermoFisher Scientific, Cat. No. 28025013) a Human Immunodeficiency Virus (HIV) reverse transcriptase (THUNDER RT polymerase, Cat. No. 7100; Varizymes, Middleton, WI). Any suitable reverse transcriptase may be used as the second enzyme. An example of an RT suitable for use in RT-LAMP is THUNDER RT polymerase, Cat. No. 7100; Varizymes, Middleton, WI).

In some embodiments, an amplification reaction mixture (e.g., a LAMP reaction mixture) comprises one or more LAMP primers and one or more additional reagents. In some embodiments, one or more (and, in some instances, each) additional reagents are in liquid form (e.g., in solution).

In certain embodiments, the one or more additional reagents comprise one or more lysis reagents. A lysis reagent generally refers to a reagent that promotes cell lysis either alone or in combination with one or more reagents and/or conditions (e.g., heating). In some embodiments, the one or more lysis reagents comprise one or more enzymes. Non-limiting examples of suitable enzymes include lysozyme, lysostaphin, zymolase, cellulase, protease, and glycanase. In some embodiments, the one or more lysis reagents comprise one or more detergents. Non-limiting examples of suitable detergents include sodium dodecyl sulphate (SDS), Sodium Hydroxide (NaOH), Proline, TWEEN (e.g., TWEEN 20, TWEEN 80), 3-[(3-cholamidopropyl) dimethylammonio]-1-propanesulfonate (CHAPS), 3-[(3-cholamidopropyl)dimethylammonio]-2-hydroxy-1-propanesulfonate (CHAPSO), TRITON X-100, and NP-40. In some embodiments, the one or more lysis reagents comprise an RNase inhibitor (e.g., a murine RNase inhibitor).

Aspects of the disclosure relate to methods of amplifying a target nucleic acid sequence using the polymerase variants of the disclosure. In some embodiments, the method comprises: a) exposing a target nucleic acid sequence to a modified DNA polymerase of the present disclosure; and providing conditions under which the modified DNA polymerase of the present disclosure amplifies the target sequence. In some embodiments, the DNA polymerase variant is a wt DNA polymerase modified to lack 5′-3′ exonuclease activity. In some embodiments, the DNA polymerase variant is a wt DNA polymerase modified to lack 5′-3′ exonuclease activity and further modified to include a histidine tag at its N-terminus. In some embodiments, the DNA polymerase variant is a variant of Sp-402 or Pb-439. In some embodiments, the Sp-402 or Pb-439 polymerases are modified to lack 5′-3′ exonuclease activity. In some embodiments, the Sp-402 or Pb-439 variant may lack 5′-3′ exonuclease activity and may further be modified to include a poly- (e.g., ten) histidine tag at its N-terminus. In some embodiments the Sp-402 or Pb-439 variant may comprise or be selected from SEQ ID NOs: 6 and 7. In some embodiments the conditions include a moderate to low temperature.

Aspects of the disclosure relate to methods of detecting a target nucleic acid sequence using the polymerase variants of the disclosure. In some embodiments, the method comprises: a) performing a nucleic acid amplification reaction configured to amplify the target nucleic acid sequence using a DNA polymerase variant of the disclosure; and b) detecting the presence or absence of the target nucleic acid sequence. In some embodiments, the DNA polymerase variant is a wt DNA polymerase modified to lack 5′-3′ exonuclease activity. In some embodiments, the DNA polymerase variant is a wt DNA polymerase modified to lack 5′-3′ exonuclease activity and further modified to include a histidine tag at its N-terminus. In some embodiments, the DNA polymerase variant is a variant of Sp-402 or Pb-439. In some embodiments, the Sp-402 or Pb-439 polymerases are modified to lack 5′-3′ exonuclease activity. In some embodiments, the Sp-402 or Pb-439 variant may lack 5′-3′ exonuclease activity and may further be modified to include a histidine tag at its N-terminus. In some embodiments the Sp-402 or Pb-439 variant may comprise or be selected from SEQ ID NOs: 6 and 7.

Devices useful for performing methods of the disclosure are also provided. The devices can comprise means for detecting the presence of a target molecule, for example components for performing one or more methods of nucleic acid extraction, amplification, and/or detection. Such components may include one or more of an amplification chamber (for example, a thermal cycler), a plate reader, a spectrophotometer, capillary electrophoresis apparatus, a chip reader, and/or robotic sample handling components. According to some embodiments, these components can be used to obtain data that reflects the presence of or is otherwise related to the target molecules in the assay being employed.

Kits useful for performing methods of the disclosure are also provided. In some embodiments, the composition, kit, or reaction mixture comprises one or more buffers (e.g., including but not limited to, Tris-HCl, Tris-AcO, KAcO, NaCl, MgCl₂, MgSO₄, and MgAcO). In some embodiments, the composition, kit or reaction mixture further comprises one or more additional components selected from, for example, nucleic acid primers, probes, dNTPs, dyes, ions, or nucleic acid controls. In some embodiments, the pH of the reaction mixture is between 8.0 and 9.0 (e.g., 8.8).

It will be understood that the variant DNA polymerases described herein may be used in any application utilizing a strand-displacing polymerase. In some embodiments, a DNA polymerase variant described herein is used in a nucleic acid sequencing method. In certain embodiments, the nucleic acid sequencing method is a long-read sequencing method. In certain embodiments, the nucleic acid sequencing method is a short-read sequencing method. In some embodiments, the nucleic acid sequencing method is a next-generation sequencing method.

### EXAMPLES

### Example 1

### Development of Variant Sp-402 and Pb-439 DNA Polymerases

Nucleic acid polymerases comprising one or more mutations relative to a full-length, wild-type Sporosarcina psychrophila (Sp-402), and Psychrobacillus sp (Pb-329) DNA polymerase were designed. As described elsewhere herein, the full-length, wild-type Sp-402 sequence used is that from Sporosarcina psychrophila (Genbank Accession No. HJF33402.1, SEQ ID NO:1) and wild-type Pb-439 used is that of Psychrobacillus sp. (Genbank Accession No. WP_298471439.1, SEQ ID NO: 2)

The Sp-402 and Pb-439 polymerases described herein have DNA-dependent DNA polymerase capabilities and possess robust strand-displacement activity.

### Optimization of Wild-Type Sp-402 and Pb-439 DNAP Sequences

The full-length, wild-type Sp-402 and Pb-439 nucleic acid polymerase (SEQ ID NO: 1 and SEQ ID NO:2) were optimized for DNA-dependent DNA polymerase and strand-displacement activity by removal of the 5′ to 3′ exonuclease domain (SEQ ID NO: 3 and SEQ ID NO: 4) and for purification by addition of an N-terminal ten-histidine tag (10×His, SEQ ID NO: 5).

First, the 5′ to 3′ exonuclease domain of full-length, wild-type Sp-402 (that Sporosarcina psychrophila (Genbank Accession No. HJF33402.1, SEQ ID NO:1)) and wild-type Pb-439 (Psychrobacillus sp. (Genbank Accession No. WP_298471439.1, SEQ ID NO: 2)), located at the N-terminus of the wild-type protein, was deleted. The 5′ to 3′ exonuclease domain which was deleted from Sp-402 is shown as SEQ ID NO: 3 and the 5′ to 3′ exonuclease domain removed from Pb-439 is shown as SEQ ID NO: 4. This deletion was made to avoid nucleolytic release of any fluorophores and quenchers conjugated to any primers that might be used in isothermal amplification assays.

Second, a ten-histidine tag was added to the N-terminus of the 5′ to 3′ exonuclease domain-deficient Sp-402 and Pb-439. The ten-histidine tag which was added is shown as SEQ ID NO: 5. This addition was made to enable traditional Ni-affinity column protein purification.

The resultant Sp-402 and Pb-439 sequence which comprises a deletion of a 5′ to 3′ exonuclease domain (SEQ ID NO: 3 and SEQ ID NO: 4) and an addition of an N-terminal ten-histidine tag (SEQ ID NO: 5) is shown as SEQ ID NO: 6 and SEQ ID NO: 7 and is referred to herein as “WT-Δexo-Sp-402 DNAP” and “WT-Δexo-Pb-439 DNAP” respectively.

### Method of Production

The Sp-402 and Pb-439 polymerase variants of the disclosure were manufactured and purified as follows. The initial plasmid of SEQ ID NO: 4 was synthesized by ATUM (Newark, CA) and then subcloned into and expressed in Lucigen's E. cloni 10G cell line (60108-1). Isolates of E. cloni 10G containing plasmids encoding Sp-402 or Pb-439 variants were stored in 50% glycerol at −80° C. before being grown in 100 mL Luria Broth (LB) with 30 μg/mL kanamycin and 0.4% glucose at 200 rpm and 30° C. overnight. The culture was then transferred to 1 L 2×LB (34 g of soy peptone, 10 g of yeast extract, and 10 of NaCl) with 30 μg/mL kanamycin and 0.4% rhamnose in baffled flasks for induction of protein expression for 18 hrs at 200 rpm and 30° C. The culture was centrifuged at 2200 rcf for 20 minutes and the pellet resuspended in 100 mM Tris-HCl pH 8.0 with 500 mM NaCl and 30 mM imidazole at a ratio of 10 mL per gram of wet cell paste. The cell suspension was sonicated using a Branson digital sonifier for 12 minutes using 15 s pulses at 50% power and 0° C. After sonication, the lysate was centrifuged for 45 minutes at 4° C. and 11000 rcf to clarify. The clarified lysate was applied to ThermoFisher HisPur Ni column equilibrated with 100 mM Tris-HCl pH 8.0 with 500 mM NaCl and 30 mM imidazole. The column was washed with 10 column volumes of 100 mM Tris-HCl pH 8.0 with 500 mM NaCl and 30 mM imidazole. The protein is eluted with 5 column volumes 100 mM Tris-HCl pH 8.0 with 500 mM NaCl and 300 mM Imidazole. The protein-elution UV-absorbance peak was collected and diluted with 100 mM Tris-HCl, pH=8 to a NaCl concentration of 100 mM and applied to a Q-sepharose column normalized with 100 mM Tris-HCl pH 8.0 with 100 mM NaCl. The column was washed with 10 volumes of 100 mM Tris-HCl pH 8.0 with 125 mM NaCl and the protein was eluted with 5 column volumes of 100 mM Tris-HCl pH=8.0 with 250 mM NaCl.

Fractions from Q-Sepharose containing the enzyme were pooled and concentrated to 10-20 mL by ultrafiltration using a Pierce Protein Concentrator with a 10 kDa molecular weight cutoff (ThermoFisher Scientific, Cat. No. 88527). The concentrate was dialyzed against 50 volumes of storage buffer (50 mM Tris-HCl pH 7.5 with 50 mM KCl, 1 mM DTT, 0.1 mM EDTA, 0.05% Tween-20, 0.05% Nonidet-P40 substitute, and 50% glycerol).

### DNA-Dependent Polymerase Activity of Sp-402 and Pb-439 Polymerases.

DNA-dependent polymerase activity for Sp-402 and Pb-439 was assessed using a single-stranded M13 primer extension assay with Bsu DNAP, Large Fragment (New England Biolabs, Cat. No. M0330 7), or Sau DNA Polymerase I, Large Fragment (Intact Genomics, Cat. No. 3595) as standards. The following 1× reaction recipe was used: 18.3 μL H₂O, 2.5 μL 10× NEBuffer™ buffer 2 (New England Biolabs, Cat. No. B7002S) for Bsu DNAP, large fragment, 10× Sau DNA Polymerase I reaction buffer (Intact Genomics, Cat No. 3592), for Sau DNA Polymerase I, large fragment, and 10×TA Buffer (Varizymes of 500 mM Tris-CH₃CO₂ [pH=8.5], 250 mM K(CH₃CO₂), and 50 mM Mg(CH₃COO)₂) for Sp-402 and Pb-439 DNA polymerases, 0.2 μL 25 mM ea. dNTPs, 1 μL 1 μg/μL ssM13mp18 (Bayou Biolabs, Cat. No. P-107), 1 μL 10 μM M13FT-41 (IDT, SEQ ID NO: 8, Table 3), 1 μL 20× dilution Quant-iT PicoGreen™ dsDNA Reagent (Invitrogen, Cat No. P7581). Reactions were incubated in a CFX-96 thermocycler at 37° C. for 40 cycles, with each cycle being a SYBR plate read every 3 seconds. The activities of each DNA-dependent DNA polymerase activities were assessed by the relative fluorescence unit (RFU) formation rates of each enzyme in a 4-step, 2-fold serial dilution from 100 ng per 25 μL.

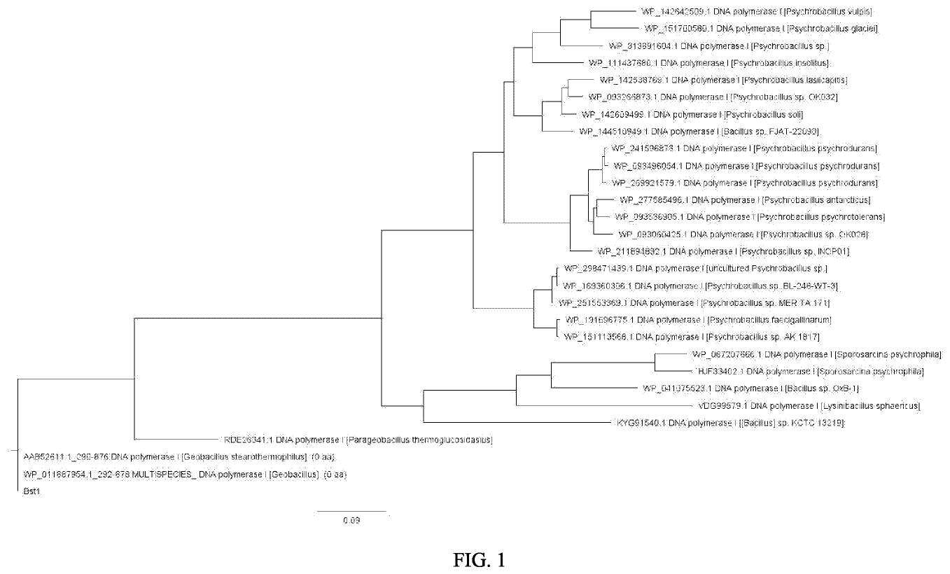

The following Sp-402 and Pb-439 DNAPs were assessed: WT-Δexo-Sp-402 DNAP (SEQ ID NO: 6) and WT-Δexo-Pb-439 DNAP (SEQ ID NO: 7). As shown in FIG. 1, both Sp-402 and Pb-439 variants possess increased strand-displacement-activity compared to Bsu DNAP, large fragment and Sau DNA polymerase I, large fragment.

### Evaluation of Sp-402 and Pb-439 DNAP and its Variants in LAMP Assay at Low Temperature

A key feature of Bst DNAP required for LAMP assays is its robust strand-displacement activity. The strong strand-displacement activity enables Bst DNAP to synthesize DNA at a constant temperature. Bst DNAPs optimal temperature range is 64-72° C. The WT-Δexo-Sp-402 DNAP and WT-Δexo-Pb-439 DNAP described in this disclosure maintained strong DNA polymerase activity of DNA targets in temperature ranges between 30-44° C. This is significant because no DNAP has been shown to have robust strand displacement at a temperature range of 37-41° C., other than phi29 DNA polymerase, which is not suitable for target specific DNA amplification. This is useful because it does not require specialized heating tools to ensure temperatures are high enough for reactions to take place and enables improved or new low temperature isothermal amplification technologies.

WT-Δexo-Pb-439 DNAP (SEQ ID NO: 7) was used in a LAMP reaction to amplify SARS-CoV-2 RNA without the addition of a secondary enzyme, reverse transcriptase polymerase. All LAMP reactions with Bsu DNAP, WT-Δexo-Sp-402 DNAP (SEQ ID NO: 6) and WT-Δexo-Pb-439 DNAP (SEQ ID NO: 7) were carried out using 10×TA buffer (Varizymes, Middleton, WI) at 37° C. Results presented in Table 6 show time to results (in minutes) for amplification of target RNA (SARS-CoV-2) at 37° C.

Briefly, the method involved preparing 10-fold serial dilutions of RNA in appropriate diluent such as water, 20 mM NaOH, and 20 mM Tris (pH 8.0)+1.5% proline, and adding each dilution (5 μL) to a 20 μL reaction mix (total reaction volume: 25 μL) containing optimized concentrations of MgSO₄, dNTP mix, DNAP (Bsu DNAP or WT-Δexo-Sp-402 DNAP or WT-Δexo-Pb-439 DNAP), target-specific primers, and intercalating dye (SYTO-82) for real-time monitoring of nucleic acid amplification. The reactions were performed in a real-time thermocycler (CFX-96, BioRad) for 170 cycles with each cycle being 30 seconds at a temperature of 37° C. Reaction kinetics were monitored in real-time by measuring the increase in fluorescence in the HEX channel associated with the accumulation of double-stranded DNA by LAMP.

As shown in Table 6, both DNAPs, Sp-402 and Pb-439, were active at low temperature (37° C.) and were able to amplify target RNA with similar efficiency as Bsu DNAP.

TABLE 2
ssM13 primer extension Primers targeting M13FT-41

| Primer | Sequence (5′ to 3′) | SEQ ID NO: |
|---|---|---|
| F3 | CGCCAGGGTTTTCCCAGTCACGAC | 8 |

TABLE 3
SARS-CoV-2 LAMP Primers

| Primer | Sequence (5′ to 3′) | SEQ ID NO: |
|---|---|---|
| F3 | GCGCGATCAAAACAACGTC | 9 |
| B3 | GGCCCAGTTCCTAGGTAG | 10 |
| FL | GTGAGAGCGGTGAACCAAG | 11 |
| BL | TCCAATTAACACCAATAGCAGTC | 12 |
| FIP | AAGGTCTTCCTTGCCATGTTGACCCAAGGTTT | 13 |
| | ACCCAATAATACTGCG | |
| BIP | AAATTCCCTCGAGGACAAGGCGTAGCTCTTC | 14 |
| | GGTAGTAGCC | |
| F3-5Phos | /5Phos/GCG CGA TCA AAA CAA CGT C | 15 |
| B3-5Phos | /5Phos/GGC CCA GTT CCT AGG TAG | 16 |
| FIP-5Phos | /5Phos/AAG GTC TTC CTT GCC ATG TTG ACC | 17 |
| | CAA GGT TTA CCC AAT AAT ACT GCG | |
| BIP-5Phos | /5Phos/AAA TTC CCT CGA GGA CAA GGC GTA | 18 |
| | GCT CTT CGG TAG TAG CC | |

TABLE 4
Polymerases and Enzymes used in assays

| Source | DNA Polymerase | Tm (° C.) |
|---|---|---|
| Intact | Sau DNAP I | 37 |
| Genomic | | |
| NEB | Bsu DNAP | 37 |
| Varizymes | PB DNAP-402 | 37 |
| | PB DNAP-439 | 37 |
| | Thunder RT | 37 |

TABLE 5
Nucleic acid sequences of Full length-WT- DNA polymerases (DNAP) 402 and 439

| SEQ | | |
|---|---|---|
| ID NO | Description | Sequence |
| 19 | SP-402 | ATGCACCATCATCATCACCATCACCACCATCACCCTAAGGAA |
| | DNAP | GCGATTGACGTCACGATTGTTAGCGAGCTGATGGACGCGGAT |
| | | CTGAGCAATGAAATGGCAATCCACGTTGAGATGATGGATGAG |
| | | AATTACCTGACGGCCGAGATCCTGGGCATCAGCCTGGCCGAC |
| | | GAGAAGAACACCCTGTTCGTCCCGCTCGAAGTGGCGAAAGAG |
| | | TGCGTCGCATTTAAGAACTGGCTGCAAAATACGGATAAGTTG |
| | | AAGTACACGAGCGACAGCAAAGCTGCGTTCGCGAGCCTGGCA |
| | | CGCCAAGGTATGGAAGCTGAGGGTTTCCAGTTCGATCTGTTG |
| | | CTGGCGACGTATATTGTTAATCCGAGCACCACGTACACCGAC |
| | | GTGGCCTCGGTGGCGGGTGAGTTTGGTTACACCGAAGTGGAG |
| | | CATGACGAAGTCATCTATGGCAAGGGTGCGAAAAAAGCCGCT |
| | | CCGGCGCAAGAAATGATTGCGGAGCATGCCGGTCGTAAAGCC |
| | | CAAGCCATCTGGAAGCTGCGTCCGGATATCGAGCAAAAGCTC |
| | | AAAGAAAACGACCAGTACGATCTGTACCACGAACTGGAACTG |
| | | CCGTTGGCGGCGATTCTGGGCAAAATGGAAACCACCGGTGTT |
| | | ATGACCGACCGTGAAACCTTGCTGACGATCGGTAGCGAACTG |
| | | ACGGCTAAACTGGCAACCTTGGAGACTACGATTTACGAAATC |
| | | GCGGGTGAGAAATTCAACATCAACAGCCCGAAGCAATTGGGC |
| | | GTTATTCTGTTTGAAAAGATCGGCCTGACGCCGATCAAAAAG |
| | | ACCAAGACCGGTTACAGCACCGCGGCAGACGTGCTGGAGAA |
| | | ATTAGAGTCTGAGCACGAAATTATTAGCCACATCCTGAACTA |
| | | TCGTCAGCTGGGTAAACTGAATAGCACCTACATTGAAGGCCT |
| | | GCTGAAAGAAATCTTCGAGGATGGTAAGATTCACACGCGTTT |
| | | CCAGCAAGCCTTGACCACGACCGGTCGCCTGTCGAGCATTAA |
| | | CCCGAATCTGCAGAACATTCCGGTGCGCCTGGAAGAGGGCCG |
| | | TAAGATTCGCGCAGCGTTCGTTCCGTCAGAGCCGGGTTGGTA |
| | | CCTGTTTGCAGCGGATTATAGCCAGATTGAGCTGAGAGTTCTG |
| | | GCTCACATCAGCCAAGACGAGAAATTGATTGAAGCATTTCGC |
| | | GAGGGTGCGGACATCCATACCAAGACCGCTATGGACGTCTTT |
| | | GGTGTGGATAATGACTCTGTCACTAGCGATATGCGCCGTGCA |
| | | GCGAAAGCCGTGAATTTCGGCATTGTTTACGGCATCAGCGAC |
| | | TACGGCCTGTCTCAGAGCCTGGGCATTACCCGTAAAGATGCA |
| | | GCCAAGTTCATCGATACGTATCTGTCCAGCTTTCCGGGTGTTA |
| | | AACAGTATATGAGCGATAGCGTAGCGGAAGCGAAGATTAAG |
| | | GGTTATGTCACCACCCTGATGAACCGTCGTCGTTATCTGCCGG |
| | | AAATCACCTCCAGCAACTTTAATCTGCGTTCCTTCGCAGAGCG |
| | | CACCGCTATGAACACTCCTATCCAGGGCAGCGCAGCGGACAT |
| | | CATCAAAAAAGCAATGATTGAGATGGCAAAGCGTCTGGAAGC |
| | | CGATGGTATGCAGACTCGTATGCTGCTGCAAGTCCACGACGA |
| | | ACTGATCTTTGAGGCGCCGGAAGATGAAATTGAGAAACTGAA |
| | | AGTGATTGTTCCAGAAGTCATGGAATCTGCGCTGGCCCTTGAC |
| | | GTGCCATTGAAAGTTGATTGGGCGTTCGGTTTATCCTGGTATG |
| | | ACACCAAGTAATAA |
| 20 | PB-439 | ATGCATCATCATCACCATCACCACCATCATCACAGCACCACC |
| | DNAP | GAGATGACCTTTGAGATTGTCGAAAATGTGAATTCCTCATTTC |
| | | TGGTGGACGAAATGTCTCTGCACCTGGAACTGTATGACGAGC |
| | | AGTACCACCGTGCTGACCTGTTGGGCATTGCGCTGAGCGACG |
| | | GCGAGAAAAGCTATTTCATTCCGGGTGAAACCGCGTTCCAGA |
| | | GCAAAGAATTTTGCGCGTGGCTGGAAGATGAGAGCAAGATCA |
| | | AATACGTTGTGGACAGCAAGGCAACTCAAGCCGTCAGCCGTA |
| | | AACATGGTGTGAGCATTGCGGGTGTTGAGTTCGATTTGACGCT |
| | | GGCGGCTTACATCGATAACGCGTCCTTAACCGCAGATGATGT |
| | | CGCGACGATTGGCAAAGAATACGGCTATTACAACGTCCTGTC |
| | | CAACGAACAGATTTACGGTAAGGGCGCGAAGAAAAGCGTGC |
| | | CGGAGCAAGAGAAACTCGCCGAGCATGCGACGCGTAAGGCTT |
| | | TGGCGCTGTGGCATCTGAAGCCGATCCTGGAGAAGAAACTGA |
| | | AAGAAAACGAACAATTCAAGCTGTACAAAGAACTGGAACTG |
| | | CCGCTGGCAAGCATTCTGGGTACGATGGAAAGCGACGGTGTC |
| | | AAAGTTGACAAACAAGTCCTGGTTGAGATGGGTCACGACCTG |
| | | GAAATTAAGCTGAAGCAAATTGAGGGTGATATTCACGCGCTG |
| | | GCGGGTGAGGCATTCAATATCAACAGCCCGAAGCAACTGGGT |
| | | GTGATCCTGTTTGAGAAACTGGGTCTGCCGCCTATCAAGAAA |
| | | ACCAAGACCGGTTATAGCACCGCGGCAGACGTGTTGGAGAAA |
| | | CTGGCGAGCGAGCACGAAATCATTGAGCAAATTCTGATGTAC |
| | | CGTCAGCTGGGCAAGCTGAATTCTACCTACATCGAAGGCCTG |
| | | CTGAAAGAAATCCACGAGTCGGATGGCAAGATTCACACGCGT |
| | | TATCAACAGGCTCTGACTACCACGGGTCGCCTGAGCAGCATT |
| | | AATCCGAATCTGCAGAATATTCCGATCCGCCTGGAAGAGGGC |
| | | CGCAAGATCCGTAAGGCCTTCGTCCCGAGCAACCCGGAATGG |
| | | GTAATGTTTGCAGCGGACTACAGCCAGATTGAGTTGCGTGTG |
| | | CTGGCTCACATGGCAGAGGACGCCAACCTGGTTGAGGCATTT |
| | | AAGAACAACATGGATATCCACACGAAAACTGCGATGGATGTT |
| | | TTTCACGTCGAAGCGGATGCAGTGGATAGCAACATGCGTCGC |
| | | GCAGCAAAAGCTGTTAATTTCGGTATCGTGTATGGCATCAGC |
| | | GACTACGGTCTTAGCCAGTCGCTGGATATTACGCGCAAAGAG |
| | | GCCGCGATTTTCATCGAGAAGTACCTGAAATCTTTCCCGGGTG |
| | | TTAAAGAATATATGGATGACAGCATCCAGACCGCGAAGCAAA |
| | | AGGGCTTTGTGACCACCATCCTGAATCGCCGTAGATATCTGCC |
| | | AGAGATCACCAGCAGCAACTTTAACTTGCGTAGCTTCGCCGA |
| | | GCGTACCGCAATGAATACGCCGATTCAGGGTTCCGCAGCCGA |
| | | CATTATCAAAAAAGCCATGATCGACATGGCTGCGCGTTTGAA |
| | | AAAAGAAAACATGCAGACCAAGATGCTGTTACAAGTTCACGA |
| | | TGAATTGATCTTCGAGGCGCCACCGGAAGAAATCGCCCTGCT |
| | | GGAAAAGATTGTCCCTGAAGTGATGGAATCCGCGATTGAGCT |
| | | GAATGTTCCGCTGAAAGTTGAGTACGCGTTCGGTAGCTCTTGG |
| | | TATGACACCAAGTAATAA |

TABLE 6
Displaying the performance of WT-Δexo-Pb-439 in RT-LAMP assay
at 37° C. The data shows amplification of SARS-CoV-2 RNA
in RT-LAMP assays with different primer sets using different diluents.

| | Diluent | | | | | | | | |
|---|---|---|---|---|---|---|---|---|---|
| | | | | 20 mM Tris (pH | | | | | |
| | Water | | | 8.0) + 1.5% Proline | | | 20 mM NaOH | | |
| Target | Primer set* | | | | | | | | |
| conc | Ctrl | P1 | P2 | Ctrl | P1 | P2 | Ctrl | P1 | P2 |
| (cp/rxn) | Time to results (minutes) | | | | | | | | |
| 10000 | 46.80 | 52.12 | 56.09 | 49.53 | 52.44 | 54.03 | 49.11 | 44.99 | 53.22 |
| 1000 | 65.49 | 59.13 | 55.16 | 56.59 | 52.77 | 63.62 | 59.94 | 60.53 | 59.98 |
| 100 | no amp | 66.45 | no amp | no amp | 61.14 | no amp | 60.63 | 63.82 | 61.22 |
| NTC | no amp | 67.15 | no amp | no amp | no amp | no amp | no amp | no amp | no amp |

*Ctrl: SEQ ID NOs: 9-14; P1: SEQ ID NOs: 11-16; P2: SEQ ID NOs: 9-12, 17, 18

One of ordinary skill in the art will readily recognize that the foregoing represents merely a detailed description of certain preferred embodiments of the present invention. Various modifications and alterations of the compositions and methods described above can readily be achieved using expertise available in the art and are within the scope of the invention.

1. A modified DNA polymerase having enhanced strand-displacement activity compared to a wild-type version of the modified DNA polymerase.

2. The modified DNA polymerase of claim 1, wherein the enhanced strand-displacement activity occurs during assays conducted at low to moderate temperatures.

3. The modified DNA polymerase of claim 1, wherein the modified DNA lacks 5′-3′ exonuclease activity.

4. The modified DNA polymerase of claim 3, wherein the modified DNA polymerase lacks at least a portion of the 5- to 3-exonuclease region of the wild-type version.

5. The modified DNA polymerase of claim 1, wherein the DNA polymerase is modified to include a histidine tag.

6. The modified DNA polymerase of claim 1, wherein the wild-type version is selected from Sp-402 (Sporosarcina psychrophila (Genbank Accession No. HJF33402.1)) or Pb-439 polymerase (Psychrobacillus sp. (Genbank Accession No. WP_298471439.1)).

7. The modified DNA polymerase of claim 6, having at least 90% sequence identity to SEQ ID NOs: 6 or 7.

8. The modified DNA polymerase of claim 6, having at least 95% sequence identity to SEQ ID NOs: 6 or 7.

9. The modified DNA polymerase of claim 6, wherein the DNA polymerase comprises SEQ ID NO: 6 or 7.

10. The modified DNA polymerase of claim 6, wherein the sequence of the DNA polymerase is SEQ ID NO: 6 or SEQ ID NO: 7.

11. A reaction mixture comprising:
a) modified DNA polymerase having enhanced strand-displacement activity compared to a wild-type version of the modified DNA polymerase; and
b) one or more reagents selected from: an enzyme that is different from the modified DNA polymerase, a buffer, nucleic acid primers, probes, dNTPs, dyes, ions, and nucleic acid controls.

12. The reaction mixture of claim 7, wherein the enzyme is a Reverse Transcriptase.

13. The reaction mixture of claim 11, wherein the enhanced strand-displacement activity of the modified DNA polymerase occurs during assays conducted at low to moderate temperatures.

14. The reaction mixture of claim 11, wherein the modified DNA polymerase lacks 5′-3′ exonuclease activity.

15. The reaction mixture of claim 14, wherein the modified DNA polymerase lacks at least a portion of the 5- to 3-exonuclease region of the wild-type version.

16. The reaction mixture of claim 11, wherein the DNA polymerase is modified to include a histidine tag.

17. The reaction mixture of claim 11, wherein the wild-type version is selected from Sp-402 (Sporosarcina psychrophila (Genbank Accession No. HJF33402.1)) or Pb-439 polymerase (Psychrobacillus sp. (Genbank Accession No. WP_298471439.1)).

18. The modified DNA polymerase of claim 17, having at least 90% sequence identity to SEQ ID NOs: 6 or 7.

19. The modified DNA polymerase of claim 17, having at least 95% sequence identity to SEQ ID NOs: 6 or 7.

20. The modified DNA polymerase of claim 17, wherein the DNA polymerase comprises SEQ ID NO: 6 or 7.

21. The modified DNA polymerase of claim 17, wherein the sequence of the DNA polymerase is SEQ ID NO: 6 or SEQ ID NO: 7.

22-51. (canceled)


SEQUENCE LISTING (20 sequences)

SEQ ID NO: 1 - protein, 874 aa, Sporosarcina psychrophila
MTTKKIVLLDGNSLAYRAFFALPLLTNDNGIHTNAVYGFTMMLQNILEEEKPTHILVAWDAGKTTFRHKTYGEYKGGRQKTPPELSEQFPYLRKLLDAYNIPQYELDQYEADDIIGTLSKTGDADGSEIVVISGDKDLTQLASSNTTVFITRKGMTDIEKYTPEHVMEKYGIEPRQIIDMKGLMGDASDNIPGVPGVGEKTALKLLVEYGSIENVYKSLDKITAKKLNQNLTENQEQAFMSKDLATIEVAAPITVSLNDLAYEGPDMEEVTSIYRELKFKTLLDKIAPQAAEEPKEAIDVTIVSELMDADLSNEMAIHVEMMDENYLTAEILGISLADEKNTLFVPLEVAKECVAFKNWLQNTDKLKYTSDSKAAFASLARQGMEAEGFQFDLLLATYIVNPSTTYTDVASVAGEFGYTEVEHDEVIYGKGAKKAAPAQEMIAEHAGRKAQAIWKLRPDIEQKLKENDQYDLYHELELPLAAILGKMETTGVMTDRETLLTIGSELTAKLATLETTIYEIAGEKFNINSPKQLGVILFEKIGLTPIKKTKTGYSTAADVLEKLESEHEIISHILNYRQLGKLNSTYIEGLLKEIFEDGKIHTRFQQALTTTGRLSSINPNLQNIPVRLEEGRKIRAAFVPSEPGWYLFAADYSQIELRVLAHISQDEKLIEAFREGADIHTKTAMDVFGVDNDSVTSDMRRAAKAVNFGIVYGISDYGLSQSLGITRKDAAKFIDTYLSSFPGVKQYMSDSVAEAKIKGYVTTLMNRRRYLPEITSSNFNLRSFAERTAMNTPIQGSAADIIKKAMIEMAKRLEADGMQTRMLLQVHDELIFEAPEDEIEKLKVIVPEVMESALALDVPLKVDWAFGLSWYDTK

SEQ ID NO: 2 - protein, 875 aa, Psychrobacillus sp.
MSSEKILLLDGNSLAYRAFFALPLLTNEHGIHTNAVYGFTMMLQKIMDEENPTHMLVAFDAGKTTFRHSTFGEYKGGRQKTPPELSEQFPYLRKLIDAYGIKRYELEMYEADDIIGTLSRQAEKKGQQVVIVSGDKDLTQLATDSTTVYITRKGITDIEKYTPEHVQEKYGLTPLQIIDMKGLMGDSSDNIPGVPGVGEKTAIKLLKEHGSVEQLYQALDSVSGAKLKEKLIANEEQAKMSKELATIEINAPIDVSLEDITYSGPNMDDVISIWKELSFKTLLEKTEYVAEEHSTTEMTFEIVENVNSSFLVDEMSLHLELYDEQYHRADLLGIALSDGEKSYFIPGETAFQSKEFCAWLEDESKIKYVVDSKATQAVSRKHGVSIAGVEFDLTLAAYIDNASLTADDVATIGKEYGYYNVLSNEQIYGKGAKKSVPEQEKLAEHATRKALALWHLKPILEKKLKENEQFKLYKELELPLASILGTMESDGVKVDKQVLVEMGHDLEIKLKQIEGDIHALAGEAFNINSPKQLGVILFEKLGLPPIKKTKTGYSTAADVLEKLASEHEIIEQILMYRQLGKLNSTYIEGLLKEIHESDGKIHTRYQQALTTTGRLSSINPNLQNIPIRLEEGRKIRKAFVPSNPEWVMFAADYSQIELRVLAHMAEDANLVEAFKNNMDIHTKTAMDVFHVEADAVDSNMRRAAKAVNFGIVYGISDYGLSQSLDITRKEAAIFIEKYLKSFPGVKEYMDDSIQTAKQKGFVTTILNRRRYLPEITSSNFNLRSFAERTAMNTPIQGSAADIIKKAMIDMAARLKKENMQTKMLLQVHDELIFEAPPEEIALLEKIVPEVMESAIELNVPLKVEYAFGSSWYDTK

SEQ ID NO: 3 - protein, 293 aa, Sporosarcina psychrophila
MTTKKIVLLDGNSLAYRAFFALPLLTNDNGIHTNAVYGFTMMLQNILEEEKPTHILVAWDAGKTTFRHKTYGEYKGGRQKTPPELSEQFPYLRKLLDAYNIPQYELDQYEADDIIGTLSKTGDADGSEIVVISGDKDLTQLASSNTTVFITRKGMTDIEKYTPEHVMEKYGIEPRQIIDMKGLMGDASDNIPGVPGVGEKTALKLLVEYGSIENVYKSLDKITAKKLNQNLTENQEQAFMSKDLATIEVAAPITVSLNDLAYEGPDMEEVTSIYRELKFKTLLDKIAPQAAEE

SEQ ID NO: 4 - protein, 293 aa, Psychrobacillus sp.
MSSEKILLLDGNSLAYRAFFALPLLTNEHGIHTNAVYGFTMMLQKIMDEENPTHMLVAFDAGKTTFRHSTFGEYKGGRQKTPPELSEQFPYLRKLIDAYGIKRYELEMYEADDIIGTLSRQAEKKGQQVVIVSGDKDLTQLATDSTTVYITRKGITDIEKYTPEHVQEKYGLTPLQIIDMKGLMGDSSDNIPGVPGVGEKTAIKLLKEHGSVEQLYQALDSVSGAKLKEKLIANEEQAKMSKELATIEINAPIDVSLEDITYSGPNMDDVISIWKELSFKTLLEKTEYVAEEH

SEQ ID NO: 5 - protein, 11 aa, Synthetic construct
MHHHHHHHHHH

SEQ ID NO: 6 - protein, 582 aa, Sporosarcina psychrophila
MPKEAIDVTIVSELMDADLSNEMAIHVEMMDENYLTAEILGISLADEKNTLFVPLEVAKECVAFKNWLQNTDKLKYTSDSKAAFASLARQGMEAEGFQFDLLLATYIVNPSTTYTDVASVAGEFGYTEVEHDEVIYGKGAKKAAPAQEMIAEHAGRKAQAIWKLRPDIEQKLKENDQYDLYHELELPLAAILGKMETTGVMTDRETLLTIGSELTAKLATLETTIYEIAGEKFNINSPKQLGVILFEKIGLTPIKKTKTGYSTAADVLEKLESEHEIISHILNYRQLGKLNSTYIEGLLKEIFEDGKIHTRFQQALTTTGRLSSINPNLQNIPVRLEEGRKIRAAFVPSEPGWYLFAADYSQIELRVLAHISQDEKLIEAFREGADIHTKTAMDVFGVDNDSVTSDMRRAAKAVNFGIVYGISDYGLSQSLGITRKDAAKFIDTYLSSFPGVKQYMSDSVAEAKIKGYVTTLMNRRRYLPEITSSNFNLRSFAERTAMNTPIQGSAADIIKKAMIEMAKRLEADGMQTRMLLQVHDELIFEAPEDEIEKLKVIVPEVMESALALDVPLKVDWAFGLSWYDTK

SEQ ID NO: 7 - protein, 583 aa, Psychrobacillus sp.
MSTTEMTFEIVENVNSSFLVDEMSLHLELYDEQYHRADLLGIALSDGEKSYFIPGETAFQSKEFCAWLEDESKIKYVVDSKATQAVSRKHGVSIAGVEFDLTLAAYIDNASLTADDVATIGKEYGYYNVLSNEQIYGKGAKKSVPEQEKLAEHATRKALALWHLKPILEKKLKENEQFKLYKELELPLASILGTMESDGVKVDKQVLVEMGHDLEIKLKQIEGDIHALAGEAFNINSPKQLGVILFEKLGLPPIKKTKTGYSTAADVLEKLASEHEIIEQILMYRQLGKLNSTYIEGLLKEIHESDGKIHTRYQQALTTTGRLSSINPNLQNIPIRLEEGRKIRKAFVPSNPEWVMFAADYSQIELRVLAHMAEDANLVEAFKNNMDIHTKTAMDVFHVEADAVDSNMRRAAKAVNFGIVYGISDYGLSQSLDITRKEAAIFIEKYLKSFPGVKEYMDDSIQTAKQKGFVTTILNRRRYLPEITSSNFNLRSFAERTAMNTPIQGSAADIIKKAMIDMAARLKKENMQTKMLLQVHDELIFEAPPEEIALLEKIVPEVMESAIELNVPLKVEYAFGSSWYDTK

SEQ ID NO: 8 - DNA, 24 bp, Synthetic construct
  source 1..24: mol_type = other DNA
cgccagggttttcccagtcacgac

SEQ ID NO: 9 - DNA, 19 bp, Synthetic construct
  source 1..19: mol_type = other DNA
gcgcgatcaaaacaacgtc

SEQ ID NO: 10 - DNA, 18 bp, Synthetic construct
  source 1..18: mol_type = other DNA
ggcccagttcctaggtag

SEQ ID NO: 11 - DNA, 19 bp, Synthetic construct
  source 1..19: mol_type = other DNA
gtgagagcggtgaaccaag

SEQ ID NO: 12 - DNA, 23 bp, Synthetic construct
  source 1..23: mol_type = other DNA
tccaattaacaccaatagcagtc

SEQ ID NO: 13 - DNA, 48 bp, Synthetic construct
  source 1..48: mol_type = other DNA
aaggtcttccttgccatgttgacccaaggtttacccaataatactgcg

SEQ ID NO: 14 - DNA, 41 bp, Synthetic construct
  source 1..41: mol_type = other DNA
aaattccctcgaggacaaggcgtagctcttcggtagtagcc

SEQ ID NO: 15 - DNA, 19 bp, Synthetic construct
  source 1..19: mol_type = other DNA
  misc_feature 1: note = 5' phosphorylation
gcgcgatcaaaacaacgtc

SEQ ID NO: 16 - DNA, 18 bp, Synthetic construct
  source 1..18: mol_type = other DNA
  misc_feature 1: note = 5' phosphorylation
ggcccagttcctaggtag

SEQ ID NO: 17 - DNA, 48 bp, Synthetic construct; identical to SEQ ID NO: 13
  source 1..48: mol_type = other DNA
  misc_feature 1: note = 5' phosphorylation

SEQ ID NO: 18 - DNA, 41 bp, Synthetic construct; identical to SEQ ID NO: 14
  source 1..41: mol_type = other DNA
  misc_feature 1: note = 5' phosphorylation

SEQ ID NO: 19 - DNA, 1782 bp, Synthetic construct
  source 1..1782: mol_type = other DNA
atgcaccatcatcatcaccatcaccaccatcaccctaaggaagcgattgacgtcacgattgttagcgagctgatggacgcggatctgagcaatgaaatggcaatccacgttgagatgatggatgagaattacctgacggccgagatcctgggcatcagcctggccgacgagaagaacaccctgttcgtcccgctcgaagtggcgaaagagtgcgtcgcatttaagaactggctgcaaaatacggataagttgaagtacacgagcgacagcaaagctgcgttcgcgagcctggcacgccaaggtatggaagctgagggtttccagttcgatctgttgctggcgacgtatattgttaatccgagcaccacgtacaccgacgtggcctcggtggcgggtgagtttggttacaccgaagtggagcatgacgaagtcatctatggcaagggtgcgaaaaaagccgctccggcgcaagaaatgattgcggagcatgccggtcgtaaagcccaagccatctggaagctgcgtccggatatcgagcaaaagctcaaagaaaacgaccagtacgatctgtaccacgaactggaactgccgttggcggcgattctgggcaaaatggaaaccaccggtgttatgaccgaccgtgaaaccttgctgacgatcggtagcgaactgacggctaaactggcaaccttggagactacgatttacgaaatcgcgggtgagaaattcaacatcaacagcccgaagcaattgggcgttattctgtttgaaaagatcggcctgacgccgatcaaaaagaccaagaccggttacagcaccgcggcagacgtgctggagaaattagagtctgagcacgaaattattagccacatcctgaactatcgtcagctgggtaaactgaatagcacctacattgaaggcctgctgaaagaaatcttcgaggatggtaagattcacacgcgtttccagcaagccttgaccacgaccggtcgcctgtcgagcattaacccgaatctgcagaacattccggtgcgcctggaagagggccgtaagattcgcgcagcgttcgttccgtcagagccgggttggtacctgtttgcagcggattatagccagattgagctgagagttctggctcacatcagccaagacgagaaattgattgaagcatttcgcgagggtgcggacatccataccaagaccgctatggacgtctttggtgtggataatgactctgtcactagcgatatgcgccgtgcagcgaaagccgtgaatttcggcattgtttacggcatcagcgactacggcctgtctcagagcctgggcattacccgtaaagatgcagccaagttcatcgatacgtatctgtccagctttccgggtgttaaacagtatatgagcgatagcgtagcggaagcgaagattaagggttatgtcaccaccctgatgaaccgtcgtcgttatctgccggaaatcacctccagcaactttaatctgcgttccttcgcagagcgcaccgctatgaacactcctatccagggcagcgcagcggacatcatcaaaaaagcaatgattgagatggcaaagcgtctggaagccgatggtatgcagactcgtatgctgctgcaagtccacgacgaactgatctttgaggcgccggaagatgaaattgagaaactgaaagtgattgttccagaagtcatggaatctgcgctggcccttgacgtgccattgaaagttgattgggcgttcggtttatcctggtatgacaccaagtaataa

SEQ ID NO: 20 - DNA, 1785 bp, Synthetic construct
  source 1..1785: mol_type = other DNA
atgcatcatcatcaccatcaccaccatcatcacagcaccaccgagatgacctttgagattgtcgaaaatgtgaattcctcatttctggtggacgaaatgtctctgcacctggaactgtatgacgagcagtaccaccgtgctgacctgttgggcattgcgctgagcgacggcgagaaaagctatttcattccgggtgaaaccgcgttccagagcaaagaattttgcgcgtggctggaagatgagagcaagatcaaatacgttgtggacagcaaggcaactcaagccgtcagccgtaaacatggtgtgagcattgcgggtgttgagttcgatttgacgctggcggcttacatcgataacgcgtccttaaccgcagatgatgtcgcgacgattggcaaagaatacggctattacaacgtcctgtccaacgaacagatttacggtaagggcgcgaagaaaagcgtgccggagcaagagaaactcgccgagcatgcgacgcgtaaggctttggcgctgtggcatctgaagccgatcctggagaagaaactgaaagaaaacgaacaattcaagctgtacaaagaactggaactgccgctggcaagcattctgggtacgatggaaagcgacggtgtcaaagttgacaaacaagtcctggttgagatgggtcacgacctggaaattaagctgaagcaaattgagggtgatattcacgcgctggcgggtgaggcattcaatatcaacagcccgaagcaactgggtgtgatcctgtttgagaaactgggtctgccgcctatcaagaaaaccaagaccggttatagcaccgcggcagacgtgttggagaaactggcgagcgagcacgaaatcattgagcaaattctgatgtaccgtcagctgggcaagctgaattctacctacatcgaaggcctgctgaaagaaatccacgagtcggatggcaagattcacacgcgttatcaacaggctctgactaccacgggtcgcctgagcagcattaatccgaatctgcagaatattccgatccgcctggaagagggccgcaagatccgtaaggccttcgtcccgagcaacccggaatgggtaatgtttgcagcggactacagccagattgagttgcgtgtgctggctcacatggcagaggacgccaacctggttgaggcatttaagaacaacatggatatccacacgaaaactgcgatggatgtttttcacgtcgaagcggatgcagtggatagcaacatgcgtcgcgcagcaaaagctgttaatttcggtatcgtgtatggcatcagcgactacggtcttagccagtcgctggatattacgcgcaaagaggccgcgattttcatcgagaagtacctgaaatctttcccgggtgttaaagaatatatggatgacagcatccagaccgcgaagcaaaagggctttgtgaccaccatcctgaatcgccgtagatatctgccagagatcaccagcagcaactttaacttgcgtagcttcgccgagcgtaccgcaatgaatacgccgattcagggttccgcagccgacattatcaaaaaagccatgatcgacatggctgcgcgtttgaaaaaagaaaacatgcagaccaagatgctgttacaagttcacgatgaattgatcttcgaggcgccaccggaagaaatcgccctgctggaaaagattgtccctgaagtgatggaatccgcgattgagctgaatgttccgctgaaagttgagtacgcgttcggtagctcttggtatgacaccaagtaataa